(19) United States
(12) Patent Application Publication
(10) Pub. No.: US 2023/0048438 A1
(43) Pub. Date: Feb. 16, 2023

(54) CROSSLINKED DEXTRAN-BASED HYDROGELS AND USES THEREOF

(71) Applicant: The Johns Hopkins University, Baltimore, MD (US)

(72) Inventors: Emily ENGLISH, Ellicott, MD (US); Matthew DAVENPORT, Baltimore, MD (US); Janty SHOGA, Baltimore, MD (US)

(73) Assignee: The Johns Hopkins University, Baltimore, MD (US)

(21) Appl. No.: 17/792,946

(22) PCT Filed: Jan. 15, 2021
(86) PCT No.: PCT/US2021/013605

§ 371 Date: Jul. 14, 2022

Related U.S. Application Data
(60) Provisional application No. 62/961,403, filed on Jan. 15, 2020.

(51) Int. Cl. A61L 26/00

(52) U.S. Cl. CPC A61L 26/0052; A61L 26/008; A61L 26/0066; A61L 2300/252; A61L 2300/258

(57) ABSTRACT

The invention is directed to compositions comprising a modified dextran and to uses thereof for the treatment of wounds in a subject or for delivering a protein, an olignonucleotide, a pharmaceutical agent, or a mixture thereof to a subject. The modified dextran in the compositions can form hydrogels via crosslinking.

### CROSS REFERENCE TO RELATED APPLICATIONS

This application claims priority to and the benefit of U.S. Provisional Application No. 62/961,403, filed Jan. 15, 2020, which is incorporated herein by reference in its entirety.

### FIELD OF THE INVENTION

The present invention is directed to biocompatible crosslinked dextran-based hydrogels, their use in methods for treating wounds, and their use as delivery vehicles for proteins, oligonucleotides, and pharmaceutical agents.

### BACKGROUND OF THE INVENTION

Polymeric hydrogels have found a broad range of pharmaceutical and biomedical applications due to their three-dimensional structure and their functional similarity to natural tissues. Various hydrogels have been prepared, based on either physical or chemical crosslinking methods. The chemical crosslinking approach to designing biodegradable hydrogels is desirable because the hydrogels are relatively easy to formulate by controlling experimental parameters, such as the type and concentration of crosslinking agents, initiator concentrations, and the ratios and concentrations of precursors.

Many different types of polymeric hydrogels have been developed since the 1950s (Kopecek, J. Nature 2002, vol. 417, pp. 388-391), and they all fall into one of two general categories of polymer: natural or synthetic. Natural polymers have gained interest over the past few decades because of their biocompatibility and the presence of biologically recognizable groups to support cellular activities (Van Tomme et al. Expert Rev. Med. Dev. 2007, vol. 4, pp. 147-164).

Among the natural polymers, dextran is a colloidal, hydrophilic, biocompatible, and nontoxic polysaccharide composed of linear α-1,6-linked D-glucopyranose residues with a low fraction of α-1,2, α-1,3 and α-1,4 linked side chains. Dextran can be biodegraded by dextranase, which exists in mammalian (including human) tissues. Dextran also has reactive hydroxyl groups (i.e. —OH) that can be modified to form hydrogels via crosslinking by photochemical and other means.

To generate chemically crosslinked dextran hydrogels, the major modification challenge is to introduce polymerizable bonds for efficient crosslinking. One approach is to incorporate vinyl groups by reacting dextran with a compound comprising a vinyl moiety such as allyl isocyanate (“AI”).

One approach to preparing dextran-based hydrogels involves the use of a synthetic polymer precursor so that the resulting hydrogels can have both synthetic and naturally occurring polymers within a single entity. One example of synthetic polymer precursors that can be coupled with dextran is polyethylene glycol (“PEG”), an amphiphilic, biocompatible but non-biodegradable polymer. PEG can be modified to incorporate polymerizable bonds, as in the case of PEG-diacrylate (“PEGDA”).

### SUMMARY OF THE INVENTION

The invention provides for compositions comprising a modified dextran, such as a dextran modified with AI (“Dextramate”). These compositions may further comprise a second crosslinkable molecule, such as poly(ethylene glycol) diacrylate (“PEGDA”), a protein, oligonucleotide, pharmaceutical agent, or mixtures thereof.

The invention also provides for compositions comprising a crosslinked modified dextran (e.g., Dextramate) or a crosslinked blend of modified dextran (e.g., Dextramate) and a second crosslinkable molecule (e.g., PEGDA), optionally further comprising a protein, oligonucleotide, pharmaceutical agent, or mixtures thereof.

The compositions of the invention may be used in methods to treat wounds in a subject or in methods for delivering a protein, olignonucleotide, pharmaceutical agent, or mixtures thereof to a subject.

### BRIEF DESCRIPTION OF THE DRAWINGS

FIG. 1 shows the preparation of an exemplary hydrogel.

FIG. 2 shows the percent swelling of hydrogels with different degrees of substitution.

FIG. 3 shows the compressive storage modulus (E′) of hydrogels with different degrees of substitution after curing for 5 minutes or 30 minutes.

FIG. 4 shows the infiltration of HL-60 s into the scaffold of hydrogels with different degrees of substitution.

FIG. 5 shows inflammatory markers (neutrophil (MPO) and macrophage (F4/80)) at Day 5 of treatment and vascularization (vessel diameter and density, CD31) at Day 14 of wound treatment in a mouse model for a control and for three hydrogels with different degrees of substitution. Dextran is labeled as D, healthy is labeled as H, wound bed is labeled as W, control healing is labeled as C, and new epithelial is labeled as E. In the model, the control consists of treating a wound by covering the wound in gauze and an adhesive dressing.

FIG. 6A shows hematoxylin and eosin (H&E) stains illustrating re-epithialization at Day 14 of wound treatment in a mouse model for a control and for three hydrogels with different degrees of substitution. Dextran is labeled as D, healthy is labeled as H, wound bed is labeled as W, control healing is labeled as C, new epithelial is labeled as E, and new follicles are labeled as F. In the model, the control consists of treating a wound by covering the wound in gauze and an adhesive dressing.

FIG. 6B shows Masson's Trichrome (MT) stain at Day 14 of wound treatment in a mouse model for a control and for three hydrogels with different degrees of substitution. A blue stain color shows collagen maturity and collagen concentration. Dextran is labeled as D, healthy is labeled as H, wound bed is labeled as W, control healing is labeled as C, new epithelial is labeled as E, and new follicles are labeled as F. In the model, the control consists of treating a wound by covering the wound in gauze and an adhesive dressing.

FIG. 6C shows hematoxylin and eosin (H&E) stain illustrating re-epithialization at Day 21 of wound treatment in a mouse model for a control and for three hydrogels with different degrees of substitution. Dextran is labeled as D, healthy is labeled as H, wound bed is labeled as W, control healing is labeled as C, new epithelial is labeled as E, and new follicles are labeled as F. In the model, the control consists of treating a wound by covering the wound in gauze and an adhesive dressing.

FIG. 6D shows Masson's Trichrome (MT) stain at Day 21 of wound treatment in a mouse model for a control and for three hydrogels with different degrees of substitution. A blue stain color shows collagen maturity and collagen concentration. Dextran is labeled as D, healthy is labeled as H, wound bed is labeled as W, control healing is labeled as C, new epithelial is labeled as E, and new follicles are labeled as F. In the model, the control consists of treating a wound by covering the wound in gauze and an adhesive dressing.

FIG. 7A shows vessel quantification (number of vessels over wound area) (CD31) at Day 14 of wound treatment in a mouse model for a control and for three hydrogels with different degrees of substitution. In the model, the control consists of treating a wound by covering the wound in gauze and an adhesive dressing.

FIG. 7B shows vessel density (total vessel area over wound area) (CD31) at Day 14 of wound treatment in a mouse model for a control and for three hydrogels with different degrees of substitution. In the model, the control consists of treating a wound by covering the wound in gauze and an adhesive dressing.

FIG. 7C shows epithelial thickness (H&E) at Day 21 of wound treatment in a mouse model for a control and for three hydrogels with different degrees of substitution. In the model, the control consists of treating a wound by covering the wound in gauze and an adhesive dressing.

FIG. 7D shows collagen mean gray value (MT) at Day 21 of wound treatment in a mouse model for a control and for three hydrogels with different degrees of substitution. In the model, the control consists of treating a wound by covering the wound in gauze and an adhesive dressing.

FIG. 7E shows the presence of neutrophils in a wound center (left bar), surrounding area (center bar) and total tissue area (right bar) after 5 days of wound treatment in a mouse model with a control and with three hydrogels having different degrees of substitution. In the model, the control consists of treating a wound by covering the wound in gauze and an adhesive dressing.

### DETAILED DESCRIPTION

Some embodiments of the current invention are described herein. In describing embodiments, specific terminology is used for the sake of clarity. However, the invention is not intended to be limited to the specific terminology so selected. A person skilled in the relevant art will recognize that other equivalent components can be employed and other methods developed without departing from the broad concepts of the current invention. All references cited herein are incorporated by reference.

### Definitions

The term “wound” as used herein refers to acute wounds, chronic wounds, diabetic ulcers, or pressure wounds. Wounds can be chronic excisional wounds, acute excisional wounds, or burn excisional wounds.

The term “modified” and variations thereof as used herein means alters. An agent that modifies a cell, substrate, or cellular environment produces a biochemical alteration in a component (e.g., polypeptide, nucleotide, or molecular component) of the cell, substrate, or cellular environment.

The term “subject” as used herein means an animal. In some embodiments, a subject may be a mammal, including, but not limited to, a human or non-human mammal, such as a bovine, equine, canine, ovine, or feline.

As used herein, the terms “treat,” treating,” “treatment,” “therapeutic” and the like refer to reducing or ameliorating a disorder or condition and/or symptoms associated therewith. It will be appreciated that, although not precluded, treating a disorder or condition does not require that the disorder, condition or symptoms associated therewith be completely eliminated.

The term “C₁-C₄ alkyl” as used herein means straight-chain, branched, or cyclic C₁-C₄ hydrocarbons which are completely saturated and hybrids thereof such as (cycloalkyl)alkyl. Examples of C₁-C₆ alkyl substituents include methyl (Me), ethyl (Et), propyl (including n-propyl (n-Pr, ⁿPr), iso-propyl (i-Pr, ⁱPr), and cyclopropyl (c-Pr, ^{c}Pr)), butyl (including n-butyl (n-Bu, ⁿBu), iso-butyl (i-Bu, ⁱBu), sec-butyl (s-Bu, ^{s}Bu), tert-butyl (t-Bu, ^{t}Bu), or cyclobutyl (c-Bu, ^{c}Bu)), and so forth.

A dextran having at least one substituted hydroxyl group can also be referred to as a “modified dextran.”

As used herein, “monomer,” “dextran monomer unit,” “dextran monomer,” and the like are used to refer to a single unit of the dextran. Dextran monomers bearing a substituent are referred to herein as “modified monomers” or “modified dextran monomers” or “modified dextran monomer units.”

### Compositions

Embodiments of the invention include compositions comprising a modified dextran. The modified dextran has at least one monomer having at least one substituted hydroxyl group, wherein the substituted hydroxyl group has the formula (III), and wherein the degree of substitution of formula (III) on the dextran is less than about 0.21; wherein formula (III) is —O₁—C(O)NR⁷—CH₂CH═CH₂ and O₁ is the oxygen atom of said substituted hydroxyl group and R⁷ is hydrogen or C₁-C₄ alkyl. In some embodiments, R⁷ is hydrogen.

In some embodiments, the degree of substitution of formula (III) on the dextran is less than about 0.21. The term “degree of substitution” (“DS”) means the average number of substituted hydroxyl groups per dextran monomer. A degree of substitution less than about 0.21 means that the number of substituted hydroxyl groups having the structure of formula (III) in the dextran, divided by the total number of monomers in the dextran is less than about 0.21.

The degree of substitution can be calculated from an ¹H NMR spectrum. For example, using the ¹H NMR spectrum of dextran modified with AI (i.e., Dextramate), the degree of substitution can be defined as the ratio of the integral of the sextet peak at 5.5-5.7 ppm to the integral of the entire dextran triplet peak at 4.7-4.9 ppm for 10 mg/mL Dextramate in D₂O measured at 400 MHz. The maximum achievable degree of substitution for dextrans is 3.00 due to the three modifiable hydroxyl groups present on unmodified dextran.

If, for example, the sum of the integrated intensities of the hydroxyl peaks is 11, and the integrated intensity of the anomeric proton is 4, the ratio would be 2.75. This value (2.75) is subtracted from the total number of hydroxyls (3), to calculate the degree of substitution (3−2.75=0.25). This also corresponds to an average of one substituted hydroxyl group for every 4 monomer units. In some embodiments, the degree of substitution is less than about 0.21.

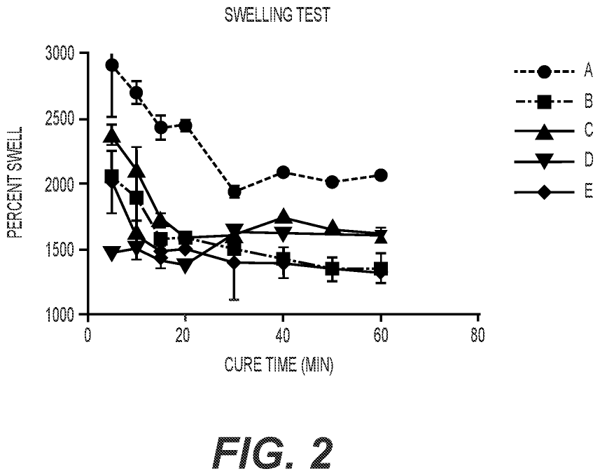

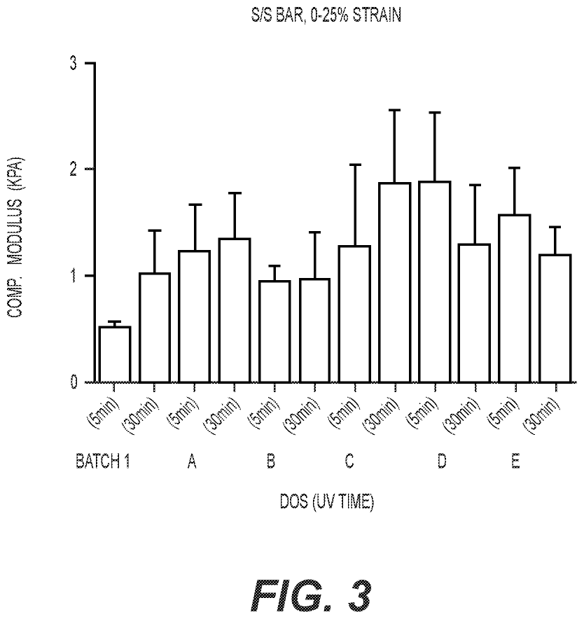

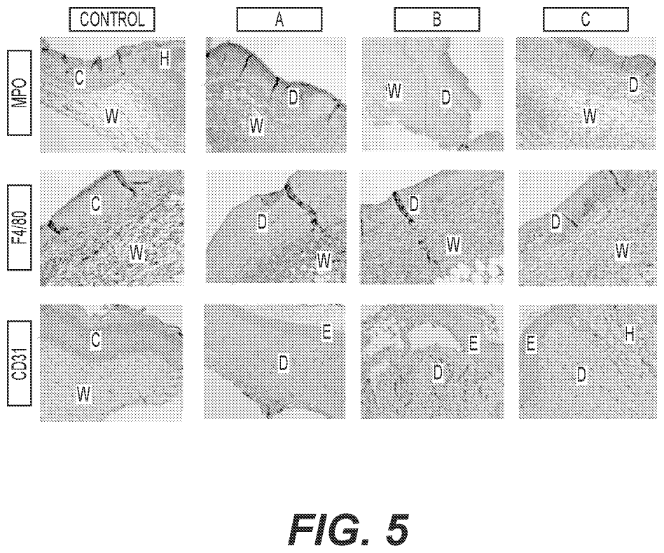

It has been found that modified dextrans that have at least one monomer having at least one substituted hydroxyl group, wherein the substituted hydroxyl group has the formula (III), and wherein the degree of substitution of formula (III) on the dextran is between about 0.15 to about 0.21 exhibit unexpected beneficial properties. For example, modified dextrans with a degree of substitution in this range exhibit superior mechanical properties and improved wound healing. FIGS. 2 and 3 show that modified dextrans with a degree of substitution in this range produce a hydrogel with a swelling ratio of greater than about 1200% and compressive storage modulus (E′) between about 1 kPa and about 2.5 kPa. Further FIGS. 5-7 show that modified dextrans with a degree of substitution in this range exhibit improved wound healing (e.g., increased blood flow back to the excision wound area, healthier epithelial thickness, and more mature and organized collagen).

In one embodiment, the degree of substitution of formula (III) on the dextran is about 0.15 to about 0.21. In a further embodiment, the degree of substitution of formula (III) on the dextran is about 0.16 to about 0.20. In a further embodiment, the degree of substitution of formula (III) on the dextran is about 0.17 to about 0.20.

In some embodiments, Dextramate is prepared by dissolving 70 kD dextran into anhydrous DMSO, adding dibutyltin dilaureate (DBTDL) and allyl isocyanate (AI) to the dextran/DMSO solution, and heating the resulting reaction mixture. Subsequently, the reaction mixture is cooled and precipitated by adding isopropyl alcohol (IPA) to the reaction mixture. The precipitate is filtered, washed, and dried under vacuum.

Some embodiments include compositions consisting essentially of the modified dextran. The modified dextran in the composition may be isolated or purified, meaning that the modified dextran has been at least partially separated from the reagents used to prepare the modified dextran. The modified dextran may be uncrosslinked, or may be crosslinked.

Other embodiments of the invention comprise the modified dextran described herein by itself, or as part of a mixture with other materials, such as a second crosslinkable molecule, a protein, an oligonucleotide, a pharmaceutical agent, or mixtures thereof.

In some embodiments, the second crosslinkable molecule is a compound comprising one or more double bonds. The one or more double bonds may be part of, for example, a vinyl, allyl, acrylate, methacrylate or alkyl acrylate structure.

In some embodiments, the second crosslinkable molecule is a polymer. As used herein, a “crosslinkable” molecule or polymer is a material bearing at least two reactive groups capable of forming a covalent bond or crosslink with the crosslinkable moiety of the dextran. Examples of crosslinkable moieties include, for example, vinyl groups, acrylate groups and, methacrylate groups. Polymers having at least two crosslinkable moieties are useable, such as PEGDA, poly(alkyleneglycol) diacrylate, and poly(alkyleneglycol) dimethacrylate. Other polymers, both degradable and nondegradable may be used. Examples include hyaluronic acid, chitosan, or poly(ester amide) polymers having crosslinkable moieties. Crosslinkable moieties other than double bonds may also be used, such as thiol containing polymers. In some embodiments, the second crosslinkable molecule is PEGDA.

In some embodiments, PEGDA may be prepared by charging 4 kD polyethylene glycol into a reaction vessel with anhydrous THF, and subsequently adding triethylamine and a solution of acryloyl chloride in THF to the reactor. The reaction mixture is heated. Subsequently, the reaction mixture is cooled and filtered. The filtrate is added to methyl tert-butyl ether (MTBE), filtered, washed, and dried under vacuum. The dried material is recrystallized in IPA, filtered, washed, and dried under vacuum.

In some embodiments, the dextran has an average molecular weight of at least about 20,000 Daltons. The dextran may have an average molecular weight of at least about 30,000 Daltons, at least about 40,000 Daltons, at least about 50,000 Daltons, or at least about 60,000 Daltons. The dextran may have an average molecular weight less than about 200,000 Daltons, less than about 150,000 Daltons, or less than about 100,000 Daltons. The dextran may have a molecular weight between any two endpoints. For example, the dextran molecule may have an average molecular weight between about 20,000 Daltons and about 200,000 Daltons, between about 20,000 Daltons and about 100,000 Daltons or between about 40,000 Daltons and about 70,000 Daltons. In one embodiment, the dextran has an average molecular weight between about 60,000 Daltons and about 80,000 Daltons. In one embodiment, the dextran has an average molecular weight of about 70,000 Daltons.

In some embodiments, the composition further comprises a protein, oligonucleotide, pharmaceutical agent, or a mixture thereof. In general, any protein, oligonucleotide, pharmaceutical agent, or a mixture thereof which may be delivered by a hydrogel may be delivered by the compositions of the present invention. Examples of proteins that may be delivered by hydrogels include bovine serum albumin (BSA) or ovalbumin. In some embodiments, the protein is a therapeutic protein, such as insulin or immunoglobulins (such as IgG). In some embodiments, the therapeutic protein is a growth factor. Examples of growth factors include vascular endothelial growth factor (VEGF), insulin growth factor (IGF), keratinocyte growth factor (KGF), stromal-cell derived factor (SDF), and angiopoetin (Ang). In some embodiments, the oligonucleotide is an antisense oligonucleotide.

In some embodiments, the composition further comprises PEGDA. In some embodiments, the PEGDA has a molecular weight of at least about 2000 Daltons, at least about 4000 Daltons, at least about 6000 Daltons, at least about 8000 Daltons, or at least about 10,000 Daltons. In some embodiments, the PEGDA has a molecular weight less than about 50,000 Daltons, less than about 20,000 Daltons, or less than about 15,000 Daltons. In one embodiment, the PEGDA has a molecular weight between about 2000 Daltons and about 6000 Daltons. In another embodiment, the PEGDA has a molecular weight between about 3000 Daltons and about 5000 Daltons. In another embodiment, the PEGDA has a molecular weight of about 4000 Daltons. The PEGDA may have a molecular weight of between any two previously disclosed endpoints. In general, larger poly(ethylene glycol) polymers are cleared more slowly from the body by the kidneys. Larger poly(ethylene glycol) may result in hydrogels with a looser structure, larger pore size, and higher swelling.

In some embodiments, the weight ratio between the dextran and PEGDA is between about 10:1 and about 1:10. In other embodiments, the weight ratio of the dextran and PEGDA is between about 80:20 and 20:80. In other embodiments, the weight of the dextran and PEGDA is between about 70:30 and 30:70. In other embodiments, the weight ratio between the dextran and PEGDA is between about 60:40 and 40:60. In some embodiments, the weight ratio between the dextran and PEGDA is about 20:80, about 40:60 or about 60:40.

In some embodiments, the composition may have a percentage (by weight) of modified dextran of greater than about 80%. In other embodiments, the percentage (by weight) of modified dextran may be greater than about 40%, greater than about 50%, greater than about 60% or greater than about 70%.

Other embodiments include compositions comprising crosslinked modified dextran as described herein. Further embodiments include compositions comprising a crosslinked blend of the modified dextran described herein and a second crosslinkable molecule, optionally further comprising a protein, oligonucleotide, pharmaceutical agent, or mixtures thereof.

In general, a crosslinking reaction occurs between the same or different molecules. As will be readily understood in the art, two modified dextrans or different portions of a single modified dextran, each of which contains at least one crosslinkable moiety, may be reacted with a non-dextran molecule or polymer having two or more crosslinkable moieties. As used herein, a crosslinkable moiety is a chemical substituent capable of reacting with another chemical substituent, forming a covalent bond or crosslink between two moieties. In the resultant structure, two dextrans or different portions of the same dextran are joined by a non-saccharide linking moiety, e.g., a PEG moiety. When crosslinked, a dextran may have multiple crosslinks to itself and/or multiple other molecules. The composition may be crosslinked between dextran molecules, or between dextran molecules and one or more other crosslinkable molecules, or between one or more other crosslinkable molecules.

In some embodiments, the second crosslinkable molecule is a polymer. Examples of crosslinkable polymers include, for example, polymers having at least two crosslinkable moieties, such as PEGDA, poly(alkyleneglycol) diacrylate, and poly(alkyleneglycol) dimethacrylate. Other crosslinkable polymers, both degradable and nondegradable, may be used. Examples include hyaluronic acid, chitosan, poly(ester amide) polymers having crosslinkable moieties, or thiol containing polymers. Thiol containing polymers may crosslink with double bond crosslinking moieties on the dextran, or thiol-containing moieties on the dextran. This chemistry may be useful for non-photocrosslinking where UV irradiation is not desirable. In some embodiments, the second crosslinkable molecule is PEGDA.

As described herein, when a second crosslinkable molecule is used, there may be a non-saccharide linking moiety between the crosslinked dextrans. For example, when the second crosslinkable molecule is PEGDA, the linking moiety is a polyethyelene glycol. In some embodiments, a crosslink can be formed between two different PEGDA molecules, between a PEGDA molecule and a modified dextran molecule (e.g. a Dextramate molecule), between two modified dextran molecules (e.g., Dextramate molecules), and/or within a single modified dextran molecule (e.g., a Dextramate molecule).

In some embodiments the crosslinked composition is a hydrogel. In other embodiments, the crosslinked composition is a hydrogel comprising a blend of dextran and PEGDA.

Embodiments of the invention include hydrogel forming compositions comprising a modified dextran in at least about 80% by weight of the composition. The modified dextran has at least one monomer having at least one substituted hydroxyl group, wherein the substituted hydroxyl group has the formula (III). Formula (III) has the structure —O₁—C(O)NR⁷—CH₂CH═CH₂ where O₁ is the oxygen atom of the substituted hydroxyl group and R⁷ is hydrogen or C₁-C₄ alkyl. The composition further includes a second crosslinkable molecule in up to about 20% by weight of the composition. In some embodiments R⁷ is H. In some embodiments, the second crosslinkable molecule is PEGDA. In other embodiments, R⁷ is H, and the second crosslinkable molecule is PEGDA.

A “hydrogel forming composition” as used herein means a composition capable of forming a solid hydrogel when crosslinked, rather than a fluid-like gel. Persons skilled in the art will generally be able to distinguish a solid hydrogel from a fluid-like hydrogel. For instance, a “solid hydrogel” is capable of maintaining its shape after crosslinking, or has sufficient structure that mechanical properties, such as the modulus may be measured. However, by way of example, and not limitation, a solid hydrogel may be considered a hydrogel having an increase in mechanical strength. Alternatively, a solid hydrogel may be a gel with a compressive storage modulus (E′) between about 250 Pa and 5000 Pa, between about 500 Pa and about 4500 Pa, between about 750 Pa and about 4000 Pa, between about 800 Pa and about 3500 Pa, or between about 900 Pa and about 3000 Pa. For example, a solid hydrogel may be a gel with a compressive storage modulus (E′) between about 1000 Pa and about 2000 Pa. A solid hydrogel may further be a gel with a gel with a shear storage modulus (G′) between about 200 Pa and about 1000 Pa, between about 300 Pa and about 900 Pa, or between about 400 Pa and about 800 Pa. A solid hydrogel may further be a gel with a compressive loss modulus (E″) of between about 100 Pa and about 500 Pa and/or a shear loss modulus (G″) of between about 10 Pa and about 100 Pa.

Storage (E′ and G′) and loss (E″ and G″) modulus values are measured using methods known in the art. For example, storage and loss modulus values may be measured using an oscillatory mechanical loading device, with loading occurring in an axial direction to the material being tested (for measurement of E′ and/or E″ modulus values) and/or in a transverse direction to the material being tested (for measurement of G′ and/or G″ modulus values). Modulus values of the hydrogels of the invention are tested under suitable parameters, such as temperature, oscillation frequency, and strain magnitude.

Suitable parameters for testing the compressive storage (E′) and compressive loss (E″) modulus values of the hydrogels of the invention are a temperature of between about 20° C. and about 27° C.; an oscillation frequency of between about 0.5 Hz to about 10 Hz; and a compressive strain magnitude of between about 2% and about 10% of the total height of the hydrogel, as determined on a TA Instruments Q800 DMA dynamic mechanical analyzer. The hydrogels of the invention used in these modulus value tests have a diameter of 8 mm and a thickness of 1.5-3.0 mm. The hydrogels of the invention are kept hydrated with excess water throughout the duration of the modulus value testing.

Suitable parameters for testing the shear storage (G′) and shear loss (G″) modulus values of the hydrogels of the invention are a temperature of between about 20° C. and about 27° C.; an oscillation frequency of between about 0.5 Hz and about 2 Hz; and a shear strain magnitude of between about 1% and about 30%, as determined on a TA Instruments AR1500ex rheometer. The hydrogels of the invention used in these modulus value tests have a diameter of 8 mm and a thickness of 1.5-3.0 mm and are compressed 10% axially. The hydrogels of the invention are kept hydrated with excess water throughout the duration of the modulus value testing.

As mentioned above, hydrogels according to the invention can be formed by crosslinking through use of, for example, chemical and photochemical means. Photochemical crosslinking can offer some advantages including reduction in the exposure to chemical initiators or other reagents, and greater control over degree of crosslinking by having direct control over exposure to light. In many cases, it is still advantageous to reduce the exposure time to UV radiation. For this reason, certain embodiments include hydrogels and hydrogel forming compositions that form solid hydrogels in a particular period of time. For instance, the compositions may form solid hydrogels in less than about 1 hour, less than about 45 minutes, less than about 30 minutes, or less than about 20 minutes using photoirradiation at 365 nm with a lamp power of about 100 W.

Other embodiments include crosslinked composition of the hydrogel forming compositions described herein.

Other embodiments include a hydrogel having at least about 80% of at least one modified dextran portion and up to about 20% PEGDA portions, where the modified dextran portion is derived from a dextran with at least one monomer having at least one substituted hydroxyl group, and the substituted hydroxyl group has the formula (III). The hydrogel is formed by photocrosslinking. As described herein, formula (III) has the structure —O₁—C(O)NR⁷—CH₂CH═CH₂ where O₁ is the oxygen atom of said substituted hydroxyl group and R⁷ is hydrogen or C₁-C₄ alkyl.

In some embodiments, a hydrogel of the invention may be prepared by mixing deionized water, Irgacure 2959, Dextramate, and PEGDA and dispensing the mixture into a mold (e.g., a silicone mold, 3×3 cm). Subsequently, the mixture is cured (e.g., at 7.0 J/cm²). After curing, deionized water is added to the cured hydrogel and the hydrogel is allowed to swell to aid in its release from the mold. The hydrogel is released from the mold and placed in a tray. The tray is sealed, and the sealed package is sterilized using known techniques, such as gamma radiation.

In some embodiments, the hydrogel forming composition may produce a hydrogel with a swelling ratio of greater than about 1200%. The swelling ratio may be determined gravimetrically by immersing a dry hydrogel sample of known weight in distilled water, and measuring the increase in weight until the weight no longer changes. The swelling ratio can then be calculated according to formula (1):

Swelling ratio=((W_{s,t}−W_{d})/W_{d})×100% (1),

where W_{d} is the weight of dry hydrogels, and W_{s,t} is the weight of swollen hydrogels at time t. The hydrogels are assumed to reach a state of swelling equilibrium when there is no difference in swelling ratio between two adjacent intervals.

In other embodiments, the composition may produce a hydrogel having a swelling ratio of greater than about 1500%, greater than about 1700% or greater than about 1900%. The hydrogels of the present invention may have a swelling ratio of greater than about 1200%, greater than about 1500, greater than about 1700%, or greater than about 1900%. In general, an increased swelling ratio results in an increased release rate of any added components such as proteins.

In some embodiments, the crosslinked compositions/hydrogels described herein further include a protein, oligonucleotide, pharmaceutical agent, or mixtures thereof. In some embodiments, the crosslinked composition comprises a protein, oligonucleotide, pharmaceutical agent, or mixtures thereof that is released from the composition over time, when present in an environment, for example an aqueous environment, having a lower concentration of the protein, oligonucleotide, pharmaceutical agent, or mixtures thereof. “Released from the composition” as used herein, means that the concentration of protein, oligonucleotide, or pharmaceutical agent in the crosslinked composition decreases. The aqueous environment may be, for instance, a buffer, such as phosphate buffered saline (PBS) or other buffer. The buffered solution may also include dextranase enzyme or dextranase enzyme may be added. The “aqueous environment” may be a bodily fluid, such as blood, plasma, saliva, tears, or lymph. The environment into which the protein, oligonucleotide, pharmaceutical agent, or mixtures thereof is released can be blood, lymph, tissue, for example an organ tissue, gastric juices, or other environment.

In some embodiments, when a crosslinked composition of modified dextran, PEGDA, and protein is incubated at 37° C. in phosphate buffered saline (PBS), less than 10% of the protein (by weight) is released from the crosslinked composition in the first 24 hours.

### Methods

Hydrogels according to the invention can be used as tissue engineering scaffolds for use in, e.g., wound repair. Exemplary embodiments of the invention include methods for delivering a protein, oligonucleotide, pharmaceutical agent, or a mixture thereof comprising administering to a subject a crosslinked composition having at least a modified dextran described herein, and a protein, oligonucleotide, pharmaceutical agent, or a mixture thereof to be delivered, wherein the protein, oligonucleotide, pharmaceutical agent, or a mixture thereof is released from the crosslinked composition over time after administration. Some embodiments include methods for delivering proteins to a subject comprising administering to said subject a crosslinked composition having at least a modified dextran described herein and said protein. In some embodiments, the protein is a therapeutic protein.

Other embodiments of the invention include methods of increasing vascular regeneration (e.g., at a wound) comprising administering a composition described herein to a subject in need thereof. In some embodiments, the composition further includes a protein that increases vascular regeneration. In some embodiments, the protein is vascular endothelial growth factor (VEGF). Other proteins that increase vascular regeneration include insulin growth factor (IGF), stromal-cell derived factor (SDF), and angiopoetin (Ang), such as angiopoetin-1 (Ang-1).

In some instances, the composition may provoke a tissue response and increase or promote vascular regeneration without any additional protein.

When a protein, such as VEGF, that increases vascular growth is administered in a composition described herein, the protein is released over time from the composition, causing increased vascular growth. Compositions of this sort may be used, for example, for the treatment of wounds or burns by applying the composition to the surface of the body. The composition may also be administered subcutaneously (i.e. below the skin) to increase vascular growth or regeneration. In other cases, the composition may be implanted at a specific location in the body, inducing vascular growth for the treatment of, for example, ischemias.

The crosslinked compositions and hydrogels described herein may be administered by any available route for administering hydrogels to a subject. The compositions may be formulated with at least one pharmaceutically acceptable carrier depending on the method of administration. The compositions may be administered, for example, orally, parenterally, subcutaneously or topically, depending on the material to be delivered to the subject and the targeted tissue.

In some embodiments, the composition may be administered to a subject as an uncrosslinked composition, followed by photochemical crosslinking to produce a hydrogel. In this way, the hydrogels may be molded to a particular shape, based on the location of administration, for example on a targeted organ.

In some embodiments, the composition is crosslinked prior to administration. The crosslinked composition may be formed in a particular shape, for example as ovoid, sphere, disc, sheet or other structure. Crosslinked compositions may be administered internally or externally.

After administration, the protein, oligonucleotide, pharmaceutical agent, or a mixture thereof is released from the crosslinked composition. The rate of release may be steady, i.e. a certain percentage, by weight, over a period of time. In other cases, a portion of the protein, oligonucleotide, pharmaceutical agent, or a mixture thereof may be released at an increasing initial rate after administration, followed by a steady-state release. In other cases, the rate of release may decrease over time after administration.

In some embodiments, a certain percentage, by weight, of the protein, oligonucleotide, pharmaceutical agent, or mixtures thereof is released in a given period of time. Embodiments of the invention include methods for sustained release of a protein, oligonucleotide, pharmaceutical agent, or mixtures thereof by administering compositions described herein. The crosslinked compositions/hydrogels of the invention stay intact for long enough to provide sufficient mechanical support for cells to migrate into the wound bed but degrade sufficiently to allow sustained release of the protein, oligonucleotide, pharmaceutical agent, or mixtures thereof. The rate of degradation of the crosslinked compositions/hydrogels of the invention can be tuned by altering their pore size, stiffness, or presence of degradation products (e.g., dextranase). In some embodiments, the protein, oligonucleotide, pharmaceutical agent, or mixtures thereof may be released over about 24 hours or more, about 48 hours or more, or about 72 hours or more.

Zero order release, where the additional component is released at a steady state is advantageous in certain circumstances. In other cases, a temporal, stimuli responsive release is desirable. The release profile may be selected based on the desired application.

The release profile may be modified, for example, by varying the ratio between modified dextran and second crosslinkable compound in the crosslinked composition, by varying the sizes of the dextran or second crosslinking compound, or by changing the degree of substitution on the dextran. Other factors such as pH may also influence the rate of release. The release profile is also influenced by the degradation rate of the crosslinked composition, which will vary from subject to subject.

There are two basic release mechanisms, diffusion, and degradation, and combinations of the two may occur. In the diffusion mechanism, a higher degree of swelling will make the diffusion faster, thus causing faster release. A hydrogel with loose structures (i.e. less crosslinked) will also make diffusion faster. A higher degree of crosslinking cause dense structures, with less diffusion. The degradation rate is dominated by the degradation polymers. For the same hydrogel system, more degradable polymer component means faster degradation, and therefore faster release. Accordingly, persons skilled in the art will be able to modify the hydrogel structure and the polymer to achieve a desired release profile.

The invention provides a method of treating wounds, comprising applying to a wound in a patient in need thereof an effective amount of the compositions of the invention. An effective amount of the compositions of the invention is an amount such that wound healing occurs faster for wounds treated with the compositions of the invention than occurs for a control, e.g., an untreated wound.

### Preparation

The modified dextrans described herein may be prepared according to methods known in the art. For example, the dextran having substituted hydroxyl groups with the structure of formula (III) with a degree of substitution of less than about 0.21 may be prepared, for example, by reacting a dextran with allylisocyanate in the presence of an activator, such as dibutyltin dilaurate (DBTDL). The degree of substitution is controlled by reducing the mole ratio of allylisocyanate to dextran to produce the desired degree of substitution. The modified dextran may be purified, for example, by precipitation, or by chromatography, such as size exclusion chromatography.

Crosslinked compositions may be prepared by crosslinking the modified dextran using any suitable chemistry, based on the crosslinking moiety. In some embodiments, where the crosslinking moiety comprises a double bond, photocrosslinking is used to crosslink the composition. The composition may further include a second crosslinkable molecule or polymer. The second crosslinkable molecule or polymer should have at least two crosslinkable moieties capable of forming crosslinks with the crosslinkable moieties of the modified dextran.

Proteins, oligonucleotides or pharmaceutical agents may be incorporated into the crosslinked composition. In some cases, the protein, oligonucleotide, pharmaceutical agent, or a mixture thereof are incorporated by soaking the crosslinked compositions in a solution containing the protein, oligonucleotide, pharmaceutical agent, or a mixture thereof. In other cases, the protein, oligonucleotide, pharmaceutical agent, or a mixture thereof may be present in a solution containing uncrosslinked modified dextran, with or without a second crosslinkable molecule. The composition is then crosslinked, for example, by photocrosslinking, to form a crosslinked composition including the protein, oligonucleotide, pharmaceutical agent, or a mixture thereof.

In exemplary embodiments, the modified dextran is a dextran having substituted hydroxyl groups with the structure of formula (III), and the second crosslinkable molecule is PEGDA.

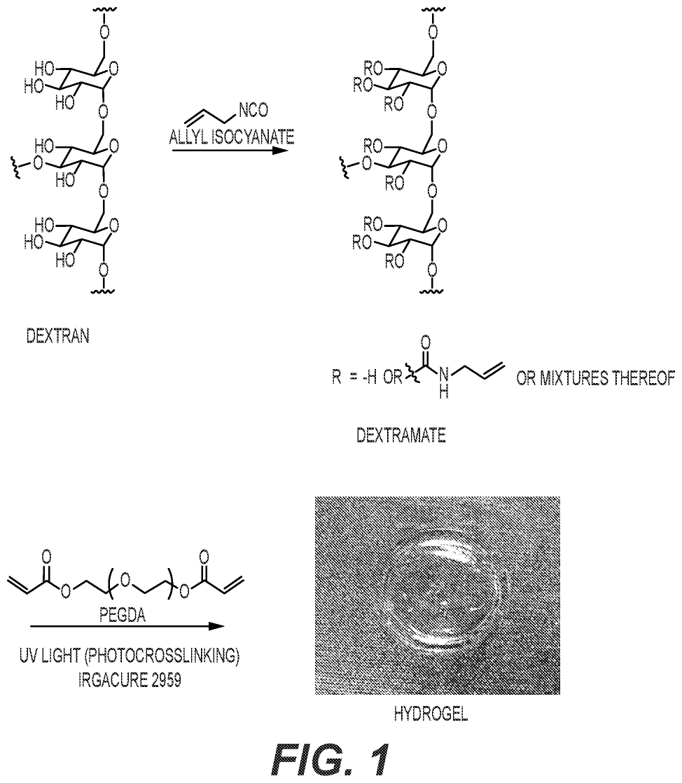

The preparation of dextran-based hydrogels is illustrated in FIG. 1. The objective of this step is to prepare the dextran-based hydrogels through the photocrosslinking of dextran-based precursors and PEGDA, using a long-wave (365 nm) UV lamp.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Terms listed in single tense also include multiple unless the context indicates otherwise.

The examples disclosed below are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

Methods for preparing, characterizing and using the compounds of this invention are illustrated in the following Examples. Starting materials are made according to procedures known in the art or as illustrated herein. The following examples are provided so that the invention might be more fully understood. These examples are illustrative only and should not be construed as limiting the invention in any way.

### EXAMPLES

### Materials

A procedure for preparing a modified dextran having at least one monomer having at least one substituted hydroxyl group, wherein the substituted hydroxyl group has the formula (III) may be found in U.S. Pat. No. 9,655,844 (“the'844 patent”), which is incorporated herein in its entirety.

Dextran (MW 70,000 Daltons, 3 g) is dried in a vacuum oven for 24 hours at 50° C. before use, and then dissolved in anhydrous dimethyl sulfoxide (DMSO) under dry nitrogen gas. Dibutyltin dilaurate (DBTDL) catalyst (1.01 mL) is injected into the solution, and then allyl isocyanate (AI) (1.64 mL) is added dropwise to the solution. The reaction is carried out for five hours at 30° C. The resulting polymer is precipitated in cold excess isopropanol. The product is further purified by dissolution and precipitation in DMSO and isopropanol, respectively. The resulting Dextramate is then dialysized (molecular weight cut off [MWCO]: 1000 Da) against distilled water for three days, lyophilized for an additional three days, and stored at 4° C. in the dark for further use.

Dextran and modified dextran are characterized for their chemical structure by FTIR and ¹H NMR using the procedure described in the '844 patent, which is incorporated herein in its entirety. Measurements of hydrogel properties—including swelling, degradation, biocompatibility, and release—and mechanical tests are performed as described in the '844 patent, which is incorporated herein in its entirety.

A procedure for preparing a PEGDA may be found in the'844 patent, which is incorporated herein in its entirety. PEG (8.0 g) is dissolved in anhydrous benzene under a nitrogen atmosphere at 40° C. and then cooled to room temperature. Triethylamine (1.19 mL) and acryloyl chloride (0.81 mL) are subsequently added. The reaction mixture is stirred for two hours at room temperature and then increased to 80° C. The resulting polymer is precipitated in hexane. It is further purified three times by dissolution and precipitation with benzene and hexane, respectively. The PEGDA is then dialysized (MWCO: 1000 Da) against distilled water for three days and then lyophilized for three days.

A procedure for preparing Dextramate/PEGDA hydrogels may be found in the'844 patent, which is incorporated herein in its entirety. Dextramate and PEGDA are dissolved at different ratios in phosphate buffered saline (PBS) containing 0.5% (w/w) 2-methyl-1-[4-(hydroxyethoxy)phenyl]-2-methyl-1-propanone (Irgacure 2959, I2959, Ciba). The mixture is pipetted into a sterile mold (50 μL volume per well, to obtain discs measuring 4 mm in diameter×2 mm thick), and photopolymerized (approximately 10 mW/cm² of UV light for ten minutes; BlakRay). The resulting hydrogels are washed in distilled water for 24 hours to remove unreacted precursors before further characterization.

For the following Examples, Dextramate samples with varying degrees of substitution (shown in Table 1) were tested:

TABLE 1

| | Measured Degree of |
|---|---|
| Sample | Substitution |
| A | 0.151 |
| B | 0.184 |
| C | 0.205 |
| D | 0.229 |
| E | 0.303 |

The degree of substitution for each of these hydrogels was determined with 1H NMR on a 400 MHz instrument, as described herein.

### Example 1

First, a baseline swell test was conducted to determine an adequate post-equilibrium time point where the swelling ratio remained unchanged over time. The selected post-equilibrium time point was 24 hours. For each of the five Dextramate samples deionized water swelling studies were conducted in triplicate over multiple UV cure times.

For all samples, the maximum swell and cure was 30 minutes, as the graph of FIG. 2 plateaus at this level. All additional testing was done for maximum cure at 30 min under direct UV. In most cases, an increase in degree of substitution resulted in a decrease in percent swelling.

Modulus values (E′, E″, G′, and G″) are measured as described herein.

### Example 2

The effect of degree of substitution on hydrogel scaffold stiffness was tested.

Dextramate sample A-E mixtures were pipetted into a sterile mold (70 μL volume per well, to obtain discs measuring 8 mm in diameter×2 mm thick). The Dextramate samples A-E were cured using UV at 5 and 30 minutes. The wavelength and the intensity of the UV light for both the 5 and 30 minutes were 365 nm and 4-5 mW/cm², respectively. The resulting hydrogels were washed in distilled water for 24 hours to remove unreacted precursors, then subsequently run through a dynamic modulus compression test under a constant load in the linear region (0-25% strain) of the stress-strain curve. This data is shown in FIG. 3.

### Example 3

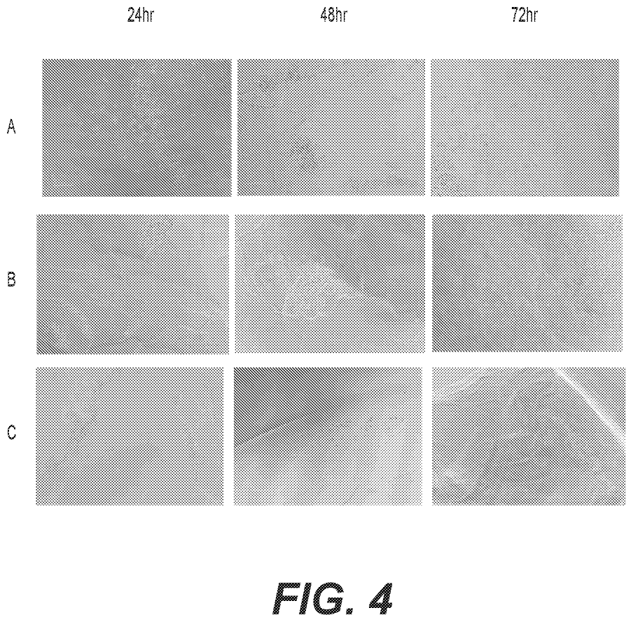

Cell degradation experiments were conducted using “DMSO differentiated” neutrophil-like cells from the HL-60 line. The experiments were conducted in triplicate over a seven day period with experimental time points at 2, 4, and 7 days. These experiments assisted in qualitatively visualizing HL-60 cell infiltration into the hydrogels with light microscopy. FIG. 4 shows cell infiltration of hydrogels resulting from samples A, B, and C (which were UV cured for 30 minutes).

### Example 4

The in vivo performance of hydrogels resulting from samples A, B, and C was tested using an excisional animal model. The study used 8 mm punch wounds on the lower dorsum of wild type c57j/Bl6. The punch wounds were covered with Curad gauze and Tegaderm. Dressings were applied every other day for two weeks.

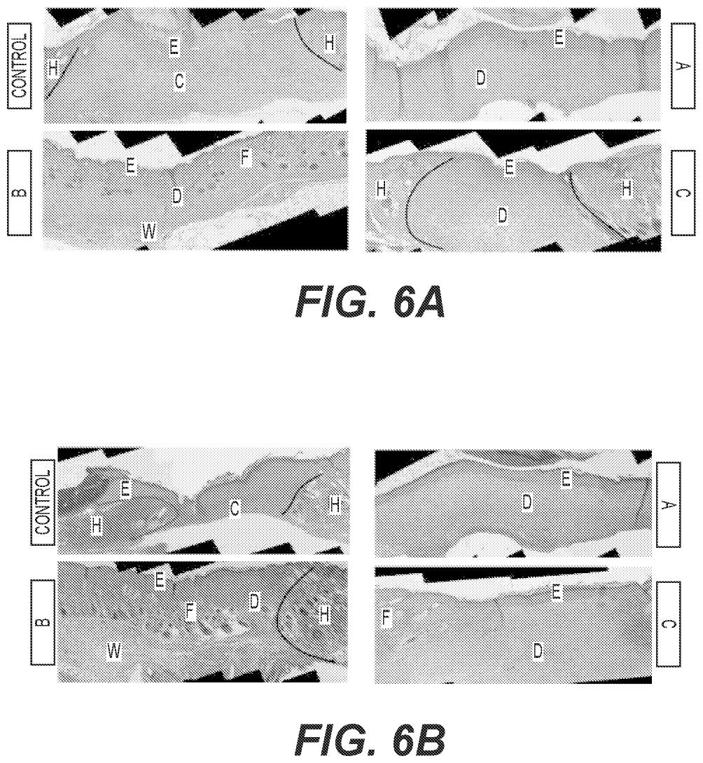

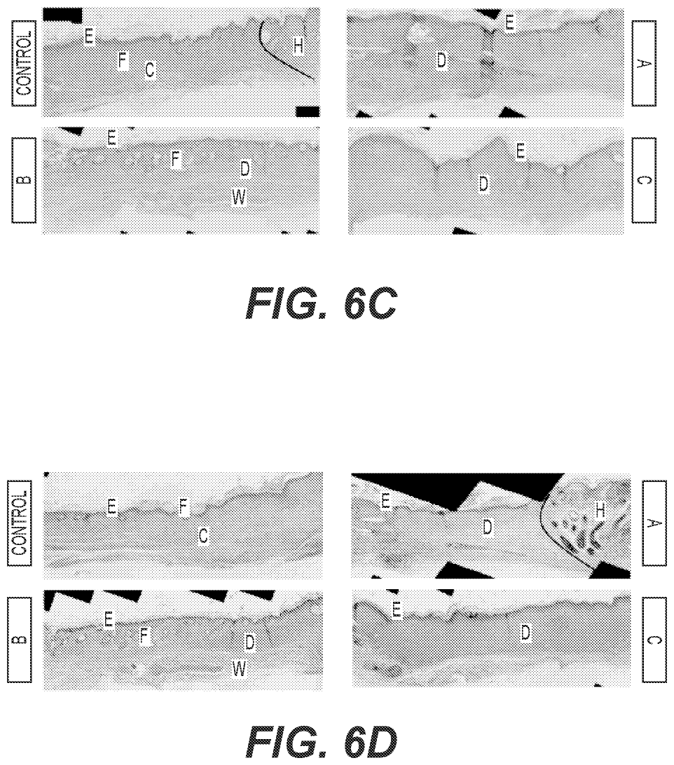

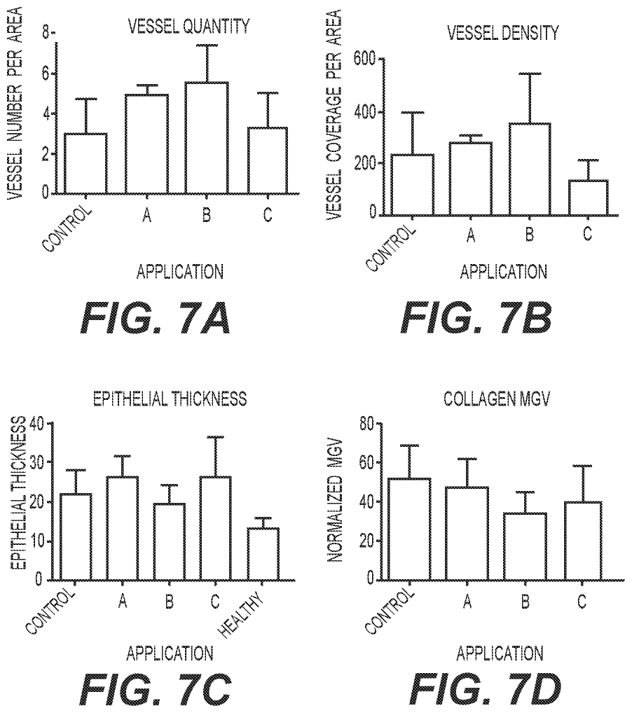

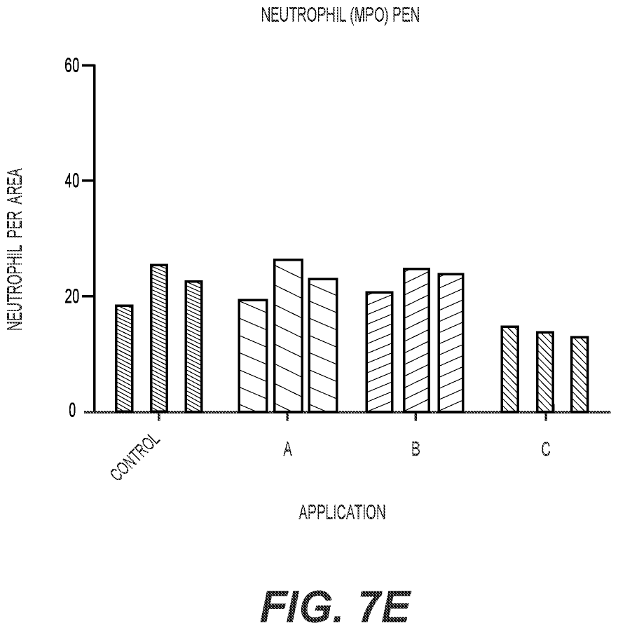

Excisions were carried out at days 5, 14, 21 and 28. Healing and hair was essentially complete by day 21. Excisions and histology at days 5 and 14 were used to determine any significant differences between application of the different hydrogels. Re-epithelization (H&E), collagen maturity and concentration (MT), vessel diameter and density (CD31), and inflammatory markers (MPO and F4/80) were observed. Representative histology is shown in FIGS. 5 and 6. A summary of these studies is shown in FIG. 7.

FIG. 5 Specialty Stains: Neutrophil (Day 5, Top); Macrophage (Day 5, Mid); and Vascularization (Day 14, Bot). FIG. 5 Labels: Dextran (D), Healthy (H), Wound Bed (W), Control Healing (C), Epithelial (E). FIGS. 6A and 6B Labels: Dextran (D), Healthy (H), Wound Bed (W), Control (C), New Epithelial (E), New Follicles (F).

Data from the study shows increased blood flow back to the excised area, a healthier epithelial thickness, and a more mature and organized collagen at day 14 in the application at the hydrogel resulting from sample B. Additionally, at day 5, inflammatory markers in the wound area were similar between the hydrogels resulting from samples A and B, and both hydrogels showed improved neutrophil penetration to the wound area compared to control.

For n=3 mice and n=6 measurements, no statistical significance between application of the different hydrogels was observed.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A composition comprising:
(a) about 80% by weight of the composition of a dextran with at least one monomer having at least one substituted hydroxyl group, wherein the substituted hydroxyl group has the formula (III); and
(b) about 20% by weight of the composition of polyethylene glycol diacrylate,
wherein formula (III) is —O₁—C(O)NR⁷—CH₂CH═CH₂, this the oxygen atom of the substituted hydroxyl group and R⁷ is hydrogen, and
wherein the degree of substitution of formula (III) on the polysaccharide is between about 0.15 and 0.21.

2. The composition of claim 1, wherein the dextran has an average molecular weight between about 60,000 Daltons and about 80,000 Daltons.

3. The composition of claim 2, wherein the dextran has an average molecular weight of about 70,000 Daltons.

4. The composition of claim 1, wherein the polyethylene glycol diacrylate has an average molecular weight between about 3000 Daltons and about 5000 Daltons.

5. The composition of claim 4, wherein the polyethylene glycol diacrylate has an average molecular weight of about 4000 Daltons.

6. The composition of claim 1, wherein the composition is crosslinked, and wherein the dextran is crosslinked by reaction with the poly(ethylene glycol) diacrylate.

7. The composition of claim 6, wherein the composition is a hydrogel.

8. The composition of claim 1, further comprising a protein, olignonucleotide, pharmaceutical agent, or a mixture thereof.

9. The composition of claim 1, wherein the degree of substitution is about 0.16 to about 0.20.

10. The composition of claim 1, wherein the degree of substitution is about 0.17 to about 0.20.

11. A method of treating wounds, comprising applying to a wound in a patient in need thereof an effective amount of the composition of claim 1.

12. The method of claim 11, wherein the composition is administered to the subject as an uncrosslinked composition, followed by photochemical crosslinking.

13. The method of claim 11, wherein the composition is crosslinked prior to administration.

14. A method of administering a protein, olignonucleotide, pharmaceutical agent, or a mixture thereof to a patient in need thereof comprising administering to the patient an effective amount of the composition of claim 8.

15. The method of claim 14, wherein the composition is administered to the subject as an uncrosslinked composition, followed by photochemical crosslinking.

16. The method of claim 14, wherein the composition is crosslinked prior to administration.
